(19) United States
(12) Patent Application Publication
(10) Pub. No.: US 2004/0006374 A1
(43) Pub. Date: Jan. 8, 2004

(54) Transcutaneous iontophoresis device using a surface electric field

(75) Inventor: Jean-Daniel Mondin, Levallois-Perret (FR)

Correspondence Address: FLEIT, KAIN, GIBBONS, GUTMAN, BONGINI, & BIANCO P.L., ONE BOCA COMMERCE CENTER, 551 NORTHWEST 77TH STREET, SUITE 111, BOCA RATON, FL, 33487, US

(73) Assignee: Guinot, Paris (FR)

(21) Appl. No.: 10/459,195

(22) Filed: Jun. 11, 2003

(30) Foreign Application Priority Data
Jun. 11, 2002 (FR) 02 07158

(51) Int. Cl. A61N 1/30; A61N 1/18

(52) U.S. Cl. 607/3; 604/20

(57) ABSTRACT

This invention relates to a transcutaneous iontophoresis device, particularly intended to activate penetration into the skin (P) of a cosmetic preparation applied to the skin, this device comprising at least two electrodes (11, 12) raised to different electrical potentials by an electricity generator (2), and the first electrode (11) being shaped such that it can be moved on the skin (P).

According to the invention, the second electrode (12) is shaped so that it can be moved about on the skin (P) at the same time as the first electrode (11) and the two electrodes (11, 12) are separated from each other by a non-zero distance (D) equal to not more than 20 centimetres.

The invention generally relates to the field of cosmetic care.

More precisely, the invention relates to a transcutaneous iontophoresis device, particularly intended to activate penetration into the skin of a cosmetic preparation applied to the skin, this device comprising at least first and second electrodes and an electricity generator, the first electrode being shaped such that it can be moved on the skin, and the electricity generator being connected to the electrodes in operation and raising them to different electrical potentials.

The transcutaneous iontophoresis technique now fairly widespread in cosmetic, and is typically implemented using a device comprising a ground electrode and an active electrode, the ground electrode being held in the hand by the patient to be treated, while the active electrode is moved on the subject's skin in an area previously covered with the cosmetic preparation.

Although this technique is statistically very satisfactory, it has some limitations related to the configuration of the electric field used in known instruments.

In particular, to the extent that this technique requires circulation of electric current throughout the body of the patient to be treated, in this known form there are sometimes some medical counter indications that in particular include wearing metallic prostheses such as tooth fillings, and the presence of electronic implants such as pacemakers.

In this context, the purpose of the invention is to propose a transcutaneous iontophoresis device with an even broader field of application than prior devices.

Consequently, the device according to the invention, conform with the generic definition given in the above preamble, is characterised essentially in that the second electrode is shaped so that it can be moved about on the skin at the same time as the first electrode and in that the first and second electrodes are separated from each other by a non-zero distance equal to not more than 20 centimetres.

With this arrangement, the electric field formed remains concentrated in the outer part of the dermis, the efficiency of the device no longer being affected by the variable distance between the electrodes in known devices, and having a strongly attenuated dependence on the different resistivity values observed between two points on the body at different points from each other on different patients.

The invention is particularly suitable for cases in which the electricity generator includes a DC power supply source.

In the preferred embodiment of the invention, at least the first electrode is composed essentially of a first electrically conducting roller that can be moved on the skin by rolling, the second electrode also possibly being composed essentially of a second electrically conducting roller, the first and second rollers then being mounted so that they can rotate freely about a common axis of rotation.

In one possible variant embodiment, the device according to the invention may include an electrically insulating shaft extending along the common axis of rotation and supporting the first and second freely rotating rollers.

An electrically insulating washer may be placed between the first and second rollers to electrically isolate them from each other.

This device may also usefully comprise a handle, the first and second rollers being installed free to rotate on one end of this handle.

The handle may then be hollow, in which case the electricity generator may be placed in the handle.

An electrical switch is advantageously installed on the handle, this switch being possibly controlled so that the generator can be selectively connected to each of the electrodes.

Finally, the device according to the invention may also comprise one or several light indicators installed on the handle, each adopting a state representative of an electrical operating state of the device.

Other characteristics and advantages of the invention will become clear from the description given below for guidance and which is in no way limitative with reference to the attached drawings in which:

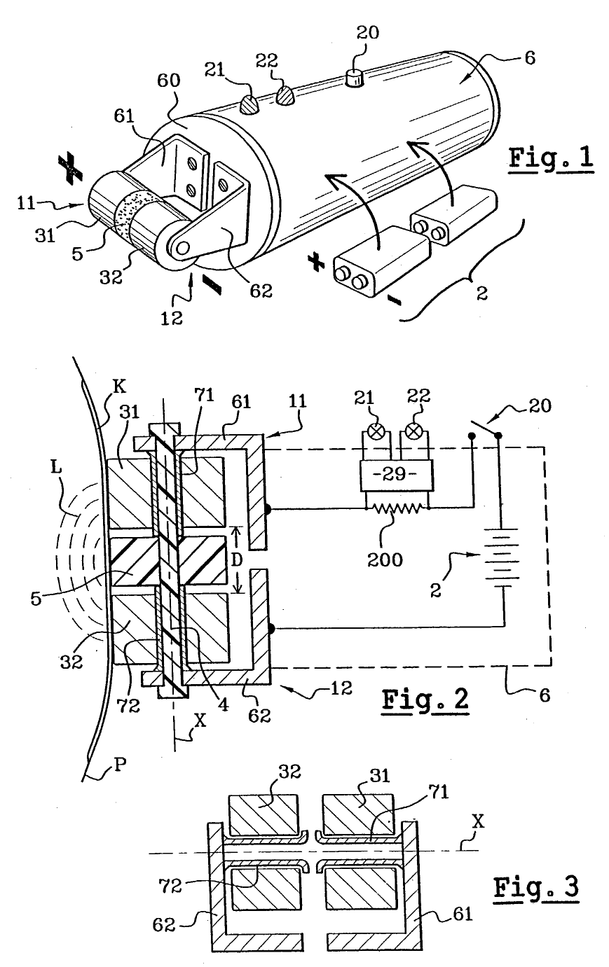

FIG. 1 is a perspective view of a device according to the invention, in which the electricity generator is shown separately;

FIG. 2 is a diagrammatic and partial sectional view of the device like that shown in FIG. 1, this figure showing the mechanical structure and the electrical arrangement of this device at the same time; and

FIG. 3 is a sectional view showing a detail of a device forming a variant embodiment of the device illustrated in FIG. 2.

As already mentioned, the invention relates to a transcutaneous iontophoresis device, intended mainly to enable a cosmetic preparation applied to the skin P of a patient (FIG. 2) to penetrate more quickly into the patient's skin.

Consequently, this device comprises two electrodes 11 and 12 and an electricity generator 2 to which these two electrodes are electrically connected during operation, in a manner known in itself, the electrodes 11 and 12 thus being raised to different electric potentials.

According to the invention, each of the electrodes 11 and 12 is shaped so that it can be moved on the skin P at the same time as the other electrode, the electrodes 11 and 12 remaining separated from each other by a nonzero distance D, but not more than approximately 20 centimetres, and typically more of the order of one centimetre.

With this arrangement, and as shown by the electric field lines L in FIG. 2, the electrodes 11 and 12 perform a local action, instead of setting up an electric current throughout the entire body as is the case at the present time with conventional instruments.

Nevertheless, the current lines L always pass not only through the skin P, but also through the cosmetic gel K spread on the skin, and thus the electric field set up actively participates in making the gel K penetrate into the skin P.

As suggested in FIG. 1, the electricity generator 2 is preferably composed of a DC current source, for example by one or several batteries.

Although each of the electrodes 11 and 12 can a priori be in several shapes, and particularly like a thin runner sliding over the skin, the electrodes 11 and 12 are advantageously composed of rollers 31 and 32.

These electrically conducting rollers, for example metallic rollers, are installed free to rotate about a common axis of rotation X and can be moved along the skin P by rolling.

For optimum handling, the device according to the invention is usefully provided with a handle 6, and more precisely with a hollow handle in which the electricity generator 2 may be fitted, particularly if it comprises batteries.

The rollers 31 and 32 may then be installed free to rotate on one end 60 of this handle 6 through metallic supports made of corresponding brackets 61 and 62, and corresponding metallic mandrels 71 and 72 fitted on these supports 61 and 62 respectively.

As can be seen in FIG. 3, the metallic mandrels 71 and 72 are at a spacing from each other such that each of the rollers 31 and 32 is only in electrical contact with the mandrel to which it is connected through its own bracket support, to the corresponding positive or negative terminal of the generator 2.

In the embodiment illustrated in FIG. 2, an electrically insulating washer 5, for example an elastomer washer, is placed between the rollers 31 and 32.

In this case, an electrically insulating shaft 4, for example made of a rigid polymer, is placed along the axis of rotation X to support the rollers 31 and 32 in free rotation and the washer 5, this shaft for example passing through the metallic mandrels 71 and 72 that are in the form of hollow cylinders.

As can be seen in FIGS. 1 and 2, an electrical switch 20 may be installed on the handle 6 so that the generator 2 can be connected to each of the electrodes 11 at 12 at the control of the user.

Moreover, a shunt 200 may be installed in series in the electrical circuit formed by the generator 2 and the electrodes 11 and 12, the electrical voltage at the terminals of this shunt being monitored by an electronic circuit 29.

Depending on the voltage at the shunt terminals 200, the electronic circuit 29 may itself control one or several light indicators such as 21 and 22 installed on the handle 6.

Under these conditions, each indicator adopts a state representing a particular electrical operating state of the device, so that the user can be kept informed that the device is working effectively at all times.

1. Transcutaneous iontophoresis device, particularly intended to activate penetration into the skin (P) of a cosmetic preparation applied to the skin, this device comprising at least first and second electrodes (11, 12) and an electricity generator (2), the first electrode (11) being shaped such that it can be moved on the skin (P), and the electricity generator (2) being connected to the electrodes (11, 12) in operation and raising them to different electrical potentials, characterised in that the second electrode (12) is shaped so that it can be moved about on the skin (P) at the same time as the first electrode (11), in that the first and second electrodes (11, 12) are composed essentially of first and second electrically conducting rollers (31, 32) that can be moved on the skin (P) by rolling, and in that the first and second electrodes (11, 12) are separated from each other by a non-zero distance (D) equal to not more than 20 centimetres

2. Transcutaneous iontophoresis device according to claim 1, characterised in that the electricity generator (2) comprises a DC current source.

3. Transcutaneous iontophoresis device according to any one of claims 1 or 2, characterised in that the first and second rollers (31, 32) are installed free to-rotate about a common axis of rotation (X).

4. Transcutaneous iontophoresis device according to claim 3, characterised in that it includes an electrically insulating shaft (4) extending along the common axis of rotation (X) and supporting the first and second freely rotating rollers (31, 32).

5. Transcutaneous iontophoresis device according to claim 4, characterised in that it comprises an electrically insulating washer (5) placed between the first and second rollers (31, 32).

6. Transcutaneous iontophoresis device according to any one of claims 4 and 5, characterised in that it comprises a handle (6), and in that the first and second rollers (31, 32) are installed free to rotate on one end (60) of this handle (6).

7. Transcutaneous iontophoresis device according to claim 6, characterised in that the handle (6) is hollow, and in that the electricity generator (2) is placed in the handle (6).

8. Transcutaneous iontophoresis device according to either of claims 6 and 7, characterised in that it comprises an electrical switch (20) installed on the handle (6), and controlled so that the generator (2) can be selectively connected to each of the electrodes (11, 12).

9. Transcutaneous iontophoresis device according to any one of claims 6 to 8, characterised in that it comprises one or several light indicators (21, 22) installed on the handle (6), and each adopting a state representative of an electrical operating state of the device.
